(19) United States
(12) Patent Application Publication
(10) Pub. No.: US 2026/0159838 A1
(43) Pub. Date: Jun. 11, 2026

(54) ANTI-CANCER TGFB2 AGENTS WITH IMMUNOTHERAPEUTICS

(71) Applicant: GMP BIOTECHNOLOGY LIMITED, Hong Kong (CN)

(72) Inventor: Vuong TRIEU, Agoura Hills, CA (US)

(21) Appl. No.: 19/403,110

(22) Filed: Nov. 27, 2025

Related U.S. Application Data
(63) Continuation of application No. PCT/US2024/032636, filed on Jun. 5, 2024.
(60) Provisional application No. 63/471,168, filed on Jun. 5, 2023.
(60) Provisional application No. 63/535,431, filed on Aug. 30, 2023.

(51) Int. Cl. C12N 15/113; A61K 31/711; A61K 38/20; A61K 39/00; A61K 45/06; A61K 47/02; A61P 35/00; C07K 16/28

(52) U.S. Cl. CPC C12N 15/1136; A61K 31/711; A61K 38/202; A61K 45/06; A61K 47/02; A61P 35/00; C07K 16/2818; C07K 16/2827; A61K 2039/505; C07K 2317/76; C12N 2310/11; C12N 2310/315

(57) ABSTRACT

This invention relates to methods for treating or ameliorating the symptoms of cancer in a human or animal subject with pharmaceutical compositions designed to promote anti-tumor effects. Exemplary synergistic pharmaceutical therapies include compositions having a combination of active agents including agents for inhibiting or suppressing expression of TGF-β2, immune checkpoint inhibitor agents, and interleukin immunotherapeutic active agents. One or more biomarkers can be used to select subjects who benefit from the method.

### SEQUENCE LISTING

This application includes a sequence listing submitted electronically as an ST.26 file created on Nov. 25, 2025, named 018988-012US1_SL.xml, which is 120,193 bytes in size.

### TECHNICAL FIELD

This invention relates to agents, compositions, and methods for use in treating or ameliorating the symptoms of cancer. Exemplary synergistic therapies include combinations of active agents for inhibiting or suppressing expression of TGF-β2, immune checkpoint inhibitor agents, and interleukin immunotherapeutic agents. One or more biomarkers can be used to select subjects who benefit from the agents, compositions, and methods.

### BACKGROUND

Cancer is a complex pathology involving multiple variant cellular pathways. Because of this complexity, many anti-cancer drugs have limited or partial therapeutic effectiveness.

Drawbacks of conventional therapies include lack of efficacy as determined by overall survival.

Further drawbacks of conventional therapies include significant unwanted side effects such as killing healthy cells in addition to killing cancer cells.

Additional drawbacks of anti-cancer agents include high toxicity at required levels of therapeutic administration.

What is needed are agents, compositions and methods for cancer diseases to increase efficacy and reduce toxicity and unwanted side effects.

Therapeutic compositions of different agents are needed to supply significant anti-tumor effects and cancer immunotherapeutic effects and which can improve efficacy, reduce side effects and reduce adverse health effects. There is a need for improved guidance for use of such compositions by using appropriate biomarkers to select synergistic effects of the agents and compositions.

There is an urgent need for new methods, agents and uses to combine strategies for cancer immunotherapy with strategies for direct anti-tumor attack for treating various cancers. The need for therapy includes therapeutic compositions which combine cancer T-cell and immunotherapies with potent anti-cancer agents.

### BRIEF SUMMARY

This invention provides agents, compositions, and methods for use in treating or ameliorating the symptoms of cancer. The compositions can promote anti-tumor effects over a range of different cancers. Synergistic pharmaceutical therapies of this invention include use of potent, direct anti-tumor agents along with cancer immunotherapeutic agents. Cancer immunotherapeutic modalities of this disclosure include combination therapies with immune checkpoint inhibitor agents and protein immunotherapeutic agents. The agents, compositions, and methods of this invention may combine strategies for cancer immunotherapy with strategies for direct anti-tumor attack for treating various cancers.

In some embodiments, compositions and methods of this invention can increase efficacy, as well as reduce toxic side effects and adverse health effects in cancer treatment.

In further embodiments, methods and therapeutic strategies of this invention can include increased guidance for successful patient outcomes using appropriate biomarkers to select synergistic effects of the compositions.

Exemplary synergistic pharmaceutical therapies include a combination of active agents including an agent for inhibiting or suppressing expression of TGF-β2, an immune checkpoint inhibitor agent, and an interleukin immunotherapeutic agent.

In further aspects, one or more biomarkers can be used to select subjects who benefit from the compositions and therapeutic methods, including IRF5 and ITGAM.

In certain embodiments, the compositions and therapeutic methods can be applied in combination with chemotherapy and other standard of care therapies for cancer.

Embodiments of this invention include the following:
A method for treating or ameliorating the symptoms of cancer in a subject in need, the method comprising: administering a composition comprising an agent for inhibiting or suppressing expression of TGF-β2; administering an immune checkpoint inhibitor; and administering an interleukin immunotherapeutic agent to the subject.
An agent for inhibiting or suppressing expression of TGF-β2 in combination with an immune checkpoint inhibitor and an interleukin immunotherapeutic active agent for use in treating or ameliorating the symptoms of cancer.
A composition comprising an agent for inhibiting or suppressing expression of TGF-β2 and a pharmaceutically acceptable carrier for use in the preparation of a medicament or for treating or ameliorating the symptoms of a cancer in a subject in combination with an immune checkpoint inhibitor and an interleukin immunotherapeutic active agent.

The method, agent or composition above, wherein the cancer is a solid cancer, a pancreatic cancer, melanoma, lung cancer, breast cancer, multiple myeloma, or colorectal cancer.

The method, agent or composition above, wherein the agent for inhibiting or suppressing expression of TGF-β2, the immune checkpoint inhibitor, and the interleukin immunotherapeutic active agent are administered concurrently, simultaneously, sequentially, or separately in time.

The method, agent or composition above, wherein the composition and agents are administered by infusion or injection.

The method, agent or composition above, wherein the agent for inhibiting or suppressing expression of TGF-β2 is selected from Table 1 or Table 2, and chemically-modified variants thereof, LNA variants thereof, gapmer variants thereof, and any combination or pooling thereof.

The method, agent or composition above, wherein the agent for inhibiting or suppressing expression of TGF-β2 is CGGCATGTCTATTTTGTA SEQ ID NO: 1 or C*G*G*C*A*T*G*T*C*T*A*T*T*T*T*G*T*A SEQ ID NO:137.

The method, agent or composition above, wherein the agent or composition comprises a carrier of sterile water for injection, saline, isotonic saline, or a combination thereof.

The method, agent or composition above, wherein the agent or composition is substantially free of excipients.

The method, agent or composition above, wherein the composition is stable for at least 14 days in carrier at 37° C.

The method, agent or composition above, wherein the immune checkpoint inhibitor is an inhibitor of PD-1, CTLA-4, or PD-L1.

The method, agent or composition above, wherein the immune checkpoint inhibitor is pembrolizumab, nivolumab, cemiplimab, spartalizumab, atezolizumab, avelumab, or durvalumab.

The method, agent or composition above, wherein the interleukin immunotherapeutic active agent is a natural IL-2, a high dose IL-2, a recombinant IL-2, or aldesleukin.

The method, agent or composition above, wherein the subject upon the administration or use has a reduced TGF-β2 expression.

The method, agent or composition above, comprising selecting the subjects who benefit from the method, agent or use using one or more biomarkers.

The method, agent or composition above, wherein the one or more biomarkers are a level of IRF5, a level of ITGAM, or a combination thereof.

The method, agent or composition above, wherein the one or more biomarkers is ITGAM and the subject is selected when expression of ITGAM is at a level above that found in a healthy patient.

The method, agent or composition above, wherein the subject after the administration or use has an increased level of IRF5.

The method, agent or composition above, wherein the subject after the administration or use has an increased level of ITGAM.

The method, agent or composition above, comprising administering a therapeutically sufficient amount of a pharmaceutical composition comprising an expression product of IRF5 or ITGAM to the subject.

The method, agent or composition above, wherein the expression product is an mRNA, a polypeptide, a protein, or fragment thereof, or combination thereof.

The method, agent or composition above, wherein the administration or use decreases mortality rate at month 6, 12, 18, 24, 30, or 36.

The method, agent or composition above, wherein the administration or use increases survival rate at month 6, 12, 18, 24, 30, or 36.

The method, agent or composition above, wherein the administration or use of the composition is combined with a standard of care treatment for cancer, wherein the standard of care treatment comprises chemotherapy or radiation therapy.

### BRIEF DESCRIPTION OF THE DRAWINGS

FIG. 1 shows a Kaplan-Meier overall survival chart obtained in a study of clinical outcomes in melanoma patients (KM Plotter). FIG. 1 shows that for use of a PD-1 checkpoint inhibitor for selected patients with high ITGAM, improved survival was shown for low TGF-β2 (logrank P=0.0039).

FIG. 2 shows a Kaplan-Meier overall survival chart obtained in a study of clinical outcomes in pancreatic cancer (KM Plotter). FIG. 2 shows that for use of IL-2 for selected patients with high ITGAM, improved survival was shown for high IL-2 (logrank P=0.034).

FIG. 3 shows a Kaplan-Meier overall survival chart obtained in a study of clinical outcomes in melanoma (KM Plotter). FIG. 3 shows that for use of a PD-1 checkpoint inhibitor for selected patients with high IRF5, improved survival was shown for low TGF-β2 (logrank P=0.00053).

FIG. 4 shows a Kaplan-Meier overall survival chart obtained in a study of clinical outcomes in cancer patients (KM Plotter). The study included multiple kinds of tumors. FIG. 4 shows that for use of a PD-1 checkpoint inhibitor, improved survival was shown for high ratio of IL-2 to TGF-β2 (IL-2/TGF-2, logrank P=0.0031).

FIG. 5 shows a Kaplan-Meier overall survival chart obtained in a study of clinical outcomes in melanoma (KM Plotter). FIG. 5 shows that for use of a PD-1 checkpoint inhibitor, improved survival was shown for high ratio of IL-2 to TGF-β2 (IL-2/TGF-β2, logrank P=3.7e-06).

FIG. 6 shows a Kaplan-Meier overall survival chart obtained in a study of clinical outcomes in melanoma patients (KM Plotter). FIG. 6 shows that for use of a CKIs, improved survival was shown for high IL-2 (logrank P=0.0015).

FIG. 7 shows a Kaplan-Meier overall survival chart obtained in a study of clinical outcomes in melanoma patients (KM Plotter). FIG. 7 shows that for use of a CKIs, improved survival was shown for low TGF-β2 (logrank P=0.006).

FIG. 8 shows a Kaplan-Meier overall survival chart obtained in a study of clinical outcomes in cancer patients (KM Plotter). FIG. 8 shows that for use of a PD-1 checkpoint inhibitor, improved survival was shown for high IL-2 (logrank P=0.007).

FIG. 9 shows a Kaplan-Meier overall survival chart obtained in a study of clinical outcomes in cancer patients (KM Plotter). FIG. 9 shows that for use of a PD-1 checkpoint inhibitor, improved survival was shown for low TGF-β2 (logrank P=0.0028).

FIG. 10 shows a Kaplan-Meier overall survival chart obtained in a study of clinical outcomes in melanoma patients (KM Plotter). The study included multiple kinds of tumors. FIG. 10 shows that for use of CKIs, improved survival was shown for high ratio of IL-2 to TGF-β2 (IL-2/TGF-β2, logrank P=3.7e-06).

FIG. 11 shows a Kaplan-Meier overall survival chart obtained in a study of clinical outcomes in cancer patients (KM Plotter). FIG. 11 shows that for use of a PD-1 checkpoint inhibitor, improved survival was shown for high ratio of IL-2 to TGF-β2 (IL-2/TGF-β2, logrank P=2.2e-08).

### DETAILED DESCRIPTION OF THE DISCLOSURE

This invention relates to methods, compositions and uses thereof for treating or ameliorating the symptoms of cancer in a human or animal subject with pharmaceutical compositions designed to promote anti-tumor effects over a range of different cancers.

Exemplary synergistic pharmaceutical therapies include compositions of various combinations of active agents including agents for inhibiting or suppressing expression of TGF-β2, checkpoint inhibitor agents, and interleukin immunotherapeutic agents.

In some embodiments, one or more biomarkers can be used to select subjects who benefit from the method, agent or use, including IRF5 and/or ITGAM. The compositions can be used in combination with chemotherapy and other standard of care therapies.

A patient or subject in need of cancer therapy as described herein may be a human or animal subject.

As used herein, the term agent can refer to one or more active compounds, a combination of active compounds, or a composition containing one or more active compounds and a carrier, and/or a solvent, and/or any number of excipients. In some embodiments, the composition may be a pharmaceutical composition. In certain embodiments, the composition may be a pharmaceutical composition containing a therapeutically effective amount of one or more active compounds. Formulations of active agents can be determined by those skilled in the art. Some examples of excipients are given in Remington's Pharmaceutical Sciences, Mack Publishing Co., Easton, Pa. 1975, and Liberman, H. A. and Lachman, L., Eds., Pharmaceutical Dosage Forms, Marcel Decker, New York, N.Y., 1980. Methods for determining a therapeutically effective amount of a compound are known in the art.

### Anti-Cancer Methods and Compositions

Embodiments of this invention include combinations of a TGF-β2-specific antisense agent, an IL-2 immunotherapy agent, and a checkpoint inhibitor.

In certain embodiments, this invention provides therapeutic combinations of one or more TGF-β2-specific antisense agents, an IL-2 immunotherapy agent, and a PD-1 checkpoint inhibitor.

In some aspects, therapeutic combinations of this invention can be used in treating or ameliorating symptoms of cancer.

In further aspects, therapeutic combinations of this invention can be used in treating or ameliorating symptoms of solid tumors.

In certain aspects, therapeutic combinations of this invention can be used in treating or ameliorating symptoms of a solid cancer, breast cancer, pancreatic cancer, melanoma, lung cancer, multiple myeloma, and colorectal cancer.

In certain aspects, therapeutic combinations of this invention can provide cytotoxicity effects which provide anti-tumor effects. For example, tumor growth may be delayed.

Embodiments of this invention provide therapeutic combinations exhibiting profound effects on survival of cancer patients. In some embodiments, tumoral levels of TGF-β2, IL-2, or the ratio IL-2/TGF-β2 may greatly impact overall survival of patients treated with immune checkpoint inhibitors.

In certain embodiments, the checkpoint inhibitors can be PD-1 checkpoint inhibitors.

In some aspects, embodiments of this invention can result in a surprisingly increased IL2/TGF-β2 ratio and improved survival for patients treated with a checkpoint inhibitor. The increase in the IL2/TGF-β2 ratio and improved survival can result from the therapeutic combination of a TGF-β2-specific antisense agent, an IL-2 immunotherapy agent, and a checkpoint inhibitor. In certain embodiments, the checkpoint inhibitor can be a PD-1 checkpoint inhibitor.

Embodiments of this invention contemplate using the therapeutic combination of a TGF-β2-specific antisense agent, an IL-2 immunotherapy agent, and a checkpoint inhibitor as a strong driver of survival for cancer patients.

In additional embodiments, overall survival for multiple kinds of cancer and tumors can be more than doubled for therapeutic combination of a TGF-β2-specific antisense agent, an IL-2 immunotherapy agent, and a checkpoint inhibitor as described herein.

In further embodiments, the number of long-term survivors for multiple kinds of cancer and tumors can be increased with a therapeutic combination of a TGF-β2-specific antisense agent, an IL-2 immunotherapy agent, and a checkpoint inhibitor as described herein.

Embodiments of this invention can demonstrate a synergy via a surprising increase in anti-cancer potency when a TGF-β2-specific antisense agent is used in combination with a checkpoint inhibitor and an interleukin immunotherapeutic agent in treating or ameliorating the symptoms of cancer.

In certain embodiments, a surprising, synergistic anti-cancer potency can be observed for treating or ameliorating the symptoms of cancer using the combination of an antisense agent for inhibiting or suppressing expression of TGF-β2, a PD-1 checkpoint inhibitor, and an IL-2 immunotherapeutic agent. This combination can be particularly effective for patients with high levels of tumor associated monocytes and/or tumor associated macrophages.

Without wishing to be bound by theory, combination of an agent for inhibiting or suppressing expression of TGF-β2 with a PD-1 checkpoint inhibitor and an IL-2 immunotherapeutic agent exhibited synergy against high levels of tumor associated monocytes and macrophages. This synergistic effect can be strongest in the combination with PD-1 checkpoint inhibitors. Because PD-1 is present in M2-type tumor associated macrophages, and antisense agents for inhibiting or suppressing expression of TGF-β2 have effects in repolarizing M2 and promoting anti-tumor effects, these two agents appear to be operable synergistically against the same target when used in combination with IL-2.

Without wishing to be bound by theory, TGF-β2 has a central role in programming M1-type tumor associated macrophages, which can exhibit anti-tumor effects. It has been demonstrated by the inventors herein that inhibiting or suppressing TGF-β2 with antisense agents has anti-tumor effects. The antisense agents may have the effect of re-programming to promote M1-type tumor associated macrophages. Such re-programming may have the effect of actively reducing and/or eliminating cancer tumors, especially when combined with an agent that is hampered by high TGF-2.

This invention includes methods for treating or ameliorating the symptoms of cancer in a human or animal subject in need, by administering a therapeutically sufficient amount of a composition comprising an agent for inhibiting or suppressing expression of TGF-β2 to the subject, administering a therapeutically sufficient amount of a composition comprising a checkpoint inhibitor to the subject, and administering a therapeutically sufficient amount of a composition comprising an interleukin immunotherapeutic agent to the subject.

Further embodiments of this invention include methods for treating or ameliorating the symptoms of cancer in a human or animal subject in need by the steps of administering a therapeutically sufficient amount of a composition comprising an agent for inhibiting or suppressing expression of TGF-β2 to the subject, administering a therapeutically sufficient amount of a composition comprising an immune checkpoint inhibitor to the subject, and administering a therapeutically sufficient amount of a composition comprising an interleukin immunotherapeutic agent to the subject.

In additional aspects, this invention includes uses of a composition comprising an agent for inhibiting or suppressing expression of TGF-β2 in the preparation of a medicament for treating or ameliorating the symptoms of a cancer in a human subject or animal in combination with a checkpoint inhibitor and an interleukin immunotherapeutic agent.

In additional aspects, an agent for inhibiting or suppressing expression of TGF-β2, an immune checkpoint inhibitor, and an interleukin immunotherapeutic agent can be administered concurrently, simultaneously, sequentially, or separately in time.

### Human TGF-β2-Specific Phosphorothioate Antisense Oligodeoxynucleotide

An antisense oligonucleotide (ASO) can be a single-stranded deoxyribonucleotide, which may be complementary to an mRNA target. The antisense therapy may downregulate a molecular target, which may be achieved by induction of RNase H endonuclease activity that cleaves the RNA-DNA heteroduplex with a significant reduction of the target gene translation. Other ASO mechanisms can include inhibition of 5′ cap formation, alteration of splicing process such as splice-switching, and steric hindrance of ribosomal activity.

Antisense therapeutic strategies can utilize single-stranded DNA oligonucleotides that inhibit protein production by mediating the catalytic degradation of a target mRNA, or by binding to sites on mRNA needed for translation. Antisense oligonucleotides can be designed to target the viral RNA genome or viral transcripts. Antisense oligonucleotides can provide an approach for identifying potential targets, and therefore represent potential therapeutics.

Antisense oligonucleotides can be small synthetic pieces of single-stranded DNA that may be 15-30 nucleotides in length. An ASO may specifically bind to a complementary DNA/RNA sequence by Watson-Crick hybridization and once bound to the target RNA, inhibit the translational processes either by inducing cleavage mechanisms or by inhibiting mRNA maturation. An ASO may selectively inhibit gene expression with specificity. Chemical modifications of DNA or RNA can be used to increase stability.

For example, modifications can be introduced in the phosphodiester bond, the sugar ring, and the backbone. ASO antiviral agents may block translational processes either by (i) ribonuclease H (RNAse H) or RNase P mediated cleavage of mRNA or (ii) by sterically (non-bonding) blocking enzymes that are involved in the target gene translation. Human TGF-β2-specific phosphorothioate antisense oligodeoxynucleotide (OT-101; AP 12009; Trabedersen), hereafter referred to as OT-101 or AP 12009, is intended to reduce the level of TGF-β2 protein in malignant gliomas, and thereby delay the progression of disease.

Antisense oligodeoxynucleotides are short strings of DNA that are designed to downregulate gene expression by interfering with the translation of a specific encoded protein at the mRNA level. OT-101 is a synthetic 18-mer phosphorothioate oligodeoxynucleotide (S-ODN) where all 3′-5′ linkages are modified to phosphorothioates. The molecular formula is C₁₇₇H₂₀₈N₆₀Na₁₇O₉₄P₁₇S₁₇ and the molecular weight 6,143 g/mol. OT-101 can be designed to be complementary to a specific sequence of human TGF-β2 mRNA following expression of the gene.

OT-101 can be supplied as a lyophilized powder in 50 mL glass vials in three different quantities. Each vial is identified by the name of the investigational product, trial number, dosing group, mode of application, quantity of OT-101 contained (in mg), total volume after dissolving (in mL) and resulting concentration (in μM), name of sponsor, name of manufacturer, batch number, vial number, storage temperature, and expiry date. The study medication can be provided in closed units, packaged separately for each concentration. The packages may contain the appropriate vial(s) and all necessary components of the application system (i.e., syringes, tube, and filter). OT-101 lyophilized powder can be dissolved in isotonic (0.9%) aqueous sodium chloride prior to use.

Examples of agents of this disclosure for inhibiting or suppressing expression of TGF-β2 include TGF-β2-specific antisense oligonucleotides given in SEQ ID NOs: 1-136 in Table 1.

TABLE 1
TGF-β2-specific antisense oligonucleotides

| SEQ | | |
|---|---|---|
| ID NO: | Ref. | ANTISENSE SEQUENCE |
| 1 | 1676-1693 | CGGCATGTCT ATTTTGTA (OT-101) |
| 2 | — | TAAAGTTCGT TTAGAGAACA G |
| 3 | — | AGCCCTGTAT ACGAC |
| 4 | — | GTAGGTAAAA ACCTAATAT |
| 5 | — | CGTTTAGAGA ACAGATCTAC |
| 6 | — | CATTGTAGAT GTCAAAAGCC |
| 7 | — | CTCCCTCATG GTGGCAGTTG A |
| 8 | — | GTTCGTTTAG AGAACAGATC |
| 9 | 178-195 | TTTGTTCCTG GATGACTC |
| 10 | 179-196 | GTTTGTTCCT GGATGACT |
| 11 | 180-197 | AGTTTGTTCC TGGATGAC |
| 12 | 181-198 | CAGTTTGTTC CTGGATGA |
| 13 | 182-199 | TCAGTTTGTT CCTGGATG |
| 14 | 183-200 | CTCAGTTTGT TCCTGGAT |
| 15 | 716-733 | TGTGTGTGTG TGCGTGTG |
| 16 | 717-734 | GTGTGTGTGT GTGCGTGT |
| 17 | 718-735 | TGTGTGTGTG TGTGCGTG |
| 18 | 719-736 | GTGTGTGTGT GTGTGCGT |
| 19 | 720-737 | TGTGTGTGTG TGTGTGCG |
| 20 | 721-738 | GTGTGTGTGT GTGTGTGC |
| 21 | 722-739 | TGTGTGTGTG TGTGTGTG |
| 22 | 723-740 | GTGTGTGTGT GTGTGTGT |
| 23 | 724-741 | TGTGTGTGTG TGTGTGTG |
| 24 | 725-742 | GTGTGTGTGT GTGTGTGT |
| 25 | 726-743 | TGTGTGTGTG TGTGTGTG |
| 26 | 727-744 | GTGTGTGTGT GTGTGTGT |
| 27 | 728-745 | TGTGTGTGTG TGTGTGTG |
| 28 | 729-746 | GTGTGTGTGT GTGTGTGT |
| 29 | 730-747 | CGTGTGTGTG TGTGTGTG |
| 30 | 731-748 | GCGTGTGTGT GTGTGTGT |
| 31 | 732-749 | TGCGTGTGTG TGTGTGTG |
| 32 | 733-750 | GTGCGTGTGT GTGTGTGT |
| 33 | 971-988 | AGTGGCGGAT CTGAACTC |
| 34 | 972-989 | GAGTGGCGGA TCTGAACT |
| 35 | 1430-1447 | GAGTGTGCTG CAGGTAGA |
| 36 | 1432-1449 | TCGAGTGTGC TGCAGGTA |
| 37 | 1433-1450 | ATCGAGTGTG CTGCAGGT |
| 38 | 1570-1587 | GTGCTGTTGT AGATGGAA |
| 39 | 1571-1588 | GGTGCTGTTG TAGATGGA |
| 40 | 1572-1589 | TGGTGCTGTT GTAGATGG |
| 41 | 1573-1590 | CTGGTGCTGT TGTAGATG |
| 42 | 1574-1591 | CCTGGTGCTG TTGTAGAT |
| 43 | 1575-1592 | CCCTGGTGCT GTTGTAGA |
| 44 | 1576-1593 | TCCCTGGTGC TGTTGTAG |
| 45 | 1577-1594 | GTCCCTGGTG CTGTTGTA |
| 46 | 1578-1595 | AGTCCCTGGT GCTGTTGT |
| 47 | 1895-1912 | CTGGGTTGGA GATGTTAA |
| 48 | 1896-1913 | GCTGGGTTGG AGATGTTA |
| 49 | 1897-1914 | CGCTGGGTTG GAGATGTT |
| 50 | 1900-1917 | TAGCGCTGGG TTGGAGAT |
| 51 | 1903-1920 | ATGTAGCGCT GGGTTGGA |
| 52 | 1945-1962 | AGCCATTCGC CTTCTGCT |
| 53 | 1994-2011 | GTCTTTATGG TGAAGCCA |
| 54 | 1995-2012 | TGTCTTTATG GTGAAGCC |
| 55 | 1996-2013 | CTGTCTTTAT GGTGAAGC |
| 56 | 1997-2014 | CCTGTCTTTA TGGTGAAG |
| 57 | 2000-2017 | GTTCCTGTCT TTATGGTG |
| 58 | 2001-2018 | GGTTCCTGTC TTTATGGT |
| 59 | 2002-2019 | AGGTTCCTGT CTTTATGG |
| 60 | 2003-2020 | CAGGTTCCTG TCTTTATG |
| 61 | 2004-2021 | CCAGGTTCCT GTCTTTAT |
| 62 | 2183-2200 | GGTCTTCCCA CTGTTTTT |
| 63 | 2194-2211 | AGGAGATGTG GGGTCTTC |
| 64 | 2195-2212 | CAGGAGATGT GGGGTCTT |
| 65 | 2241-2258 | GGTTGGTCTG TTGTGACT |
| 66 | 2242-2259 | CGGTTGGTCT GTTGTGAC |
| 67 | 2243-2260 | CCGGTTGGTC TGTTGTGA |
| 68 | 2522-2539 | GAGAATGGTT AGAGGTTC |
| 69 | 2525-2542 | GTAGAGAATG GTTAGAGG |
| 70 | 2542-2559 | GGTGTTTTGC CAATGTAG |
| 71 | 2543-2560 | GGGTGTTTTG CCAATGTA |
| 72 | 2544-2561 | TGGGTGTTTT GCCAATGT |
| 73 | 2666-2683 | CATCATCGTT GTCGTCGT |
| 74 | 2979-2996 | GAACGGTACG TACAGCAA |
| 75 | 2980-2997 | GGAACGGTAC GTACAGCA |
| 76 | 2981-2998 | AGGAACGGTA CGTACAGC |
| 77 | 2982-2999 | TAGGAACGGT ACGTACAG |
| 78 | 2983-3000 | ATAGGAACGG TACGTACA |
| 79 | 2984-3001 | GATAGGAACG GTACGTAC |
| 80 | 2985-3002 | GGATAGGAAC GGTACGTA |
| 81 | 2986-3003 | GGGATAGGAA CGGTACGT |
| 82 | 3029-3046 | GGGTGCCTAT TGCATAGC |
| 83 | 3030-3047 | AGGGTGCCTA TTGCATAG |
| 84 | 3031-3048 | AAGGGTGCCT ATTGCATA |
| 85 | 3032-3049 | GAAGGGTGCC TATTGCAT |
| 86 | 3033-3050 | GGAAGGGTGC CTATTGCA |
| 87 | 3035-3052 | TGGGAAGGGT GCCTATTG |
| 88 | 3036-3053 | ATGGGAAGGG TGCCTATT |
| 89 | 3037-3054 | AATGGGAAGG GTGCCTAT |
| 90 | 3038-3055 | GAATGGGAAG GGTGCCTA |
| 91 | 3039-3056 | AGAATGGGAA GGGTGCCT |
| 92 | 3040-3057 | AAGAATGGGA AGGGTGCC |
| 93 | 3041-3058 | TAAGAATGGG AAGGGTGC |
| 94 | 3042-3059 | GTAAGAATGG GAAGGGTG |
| 95 | 3043-3060 | AGTAAGAATG GGAAGGGT |
| 96 | 3044-3061 | GAGTAAGAAT GGGAAGGG |
| 97 | 3259-3276 | CAGACTTTCT CGGTCATA |
| 98 | 3260-3277 | GCAGACTTTC TCGGTCAT |
| 99 | 3261-3278 | TGCAGACTTT CTCGGTCA |
| 100 | 3262-3279 | ATGCAGACTT TCTCGGTC |
| 101 | 3263-3280 | AATGCAGACT TTCTCGGT |
| 102 | 3264-3281 | TAATGCAGAC TTTCTCGG |
| 103 | 4281-4298 | GACCTGGACT TTTTTCCC |
| 104 | 4282-4299 | TGACCTGGAC TTTTTTCC |
| 105 | 4283-4300 | CTGACCTGGA CTTTTTTC |
| 106 | 4284-4301 | GCTGACCTGG ACTTTTTT |
| 107 | 4467-4484 | CTGCAATGAT GTGGCAAA |
| 108 | 4468-4485 | TCTGCAATGA TGTGGCAA |
| 109 | 4469-4486 | TTCTGCAATG ATGTGGCA |
| 110 | 4470-4487 | CTTCTGCAAT GATGTGGC |
| 111 | 5062-5079 | GCTGCCCACT TGCATACT |
| 112 | 5569-5586 | GTTGGCAGAA CATAGAAC |
| 113 | 5570-5587 | CGTTGGCAGA ACATAGAA |
| 114 | 5571-5588 | GCGTTGGCAG AACATAGA |
| 115 | 5616-5633 | ATGGGGCTAC AGGGGATA |
| 116 | 5617-5634 | TATGGGGCTA CAGGGGAT |
| 117 | 5618-5635 | TTATGGGGCT ACAGGGGA |
| 118 | 5620-5637 | AGTTATGGGG CTACAGGG |
| 119 | 5621-5638 | AAGTTATGGG GCTACAGG |
| 120 | 5622-5639 | CAAGTTATGG GGCTACAG |
| 121 | 5623-5640 | CCAAGTTATG GGGCTACA |
| 122 | 5624-5641 | TCCAAGTTAT GGGGCTAC |
| 123 | 5625-5642 | ATCCAAGTTA TGGGGCTA |
| 124 | 5626-5643 | TATCCAAGTT ATGGGGCT |
| 125 | 5627-5644 | CTATCCAAGT TATGGGGC |
| 126 | 5759-5776 | ATTGGAGGAA ATAGGGTG |
| 127 | 5783-5800 | GTCTTGTAGG TAGCAGCC |
| 128 | 5784-5801 | GGTCTTGTAG GTAGCAGC |
| 129 | 5785-5802 | TGGTCTTGTA GGTAGCAG |
| 130 | 5786-5803 | CTGGTCTTGT AGGTAGCA |
| 131 | 5787-5804 | TCTGGTCTTG TAGGTAGC |
| 132 | 5788-5805 | GTCTGGTCTT GTAGGTAG |
| 133 | 5789-5806 | AGTCTGGTCT TGTAGGTA |
| 134 | 5790-5807 | GAGTCTGGTC TTGTAGGT |
| 135 | 5791-5808 | GGAGTCTGGT CTTGTAGG |
| 136 | 5792-5809 | AGGAGTCTGG TCTTGTAG |

The sequences of Table 1 can be chemically-modified to provide active variants thereof, LNA variants thereof, as well as gapmer variants thereof, as known in the art. The sequences of Table 1 can be used in any combination as active agents, such as pooling combinations.

It is understood that additional antisense oligonucleotides of this disclosure can be constructed based on the TGF-β2 gene sequence.

In some embodiments, an agent of antisense sequences can be gapmers formed by adding 1 to 5 protected ribo-nucleotides on each flank of the phosphorothioate deoxy-nucleotide sequences in Table 1. For example, the ribo-nucleotides can be protected with 2′-OMe, 2′-OEt, or 2′-O-MOE substituents, or with LNA, cMOE, or cEt bridges, as well as phosphorothioate linkages.

In some embodiments, an agent of antisense sequences can be a n-M-n RNA (2′-OMe)*-DNA*-RNA (2′-OMe)*gapmer, where n is from 3-7 and M is from 6-12. In certain embodiments, the gapmer can be a 3-10-3 or 5-10-5 LNA*-DNA*-LNA*or cEt*-DNA*-cEt*gapmer (* designates phosphorothioate linkages).

Examples of agents of this disclosure for inhibiting or suppressing expression of TGF-β2 include TGF-β2-specific phosphorothioate antisense oligonucleotides given in SEQ ID NOs: 137-144 in Table 2, based on the sequences in Table 1.

TABLE 2
TGF-β2-specific phosphorothioate
antisense oligonucleotides

| SEQ | | |
|---|---|---|
| ID NO: | Ref. | ANTISENSE SEQUENCE |
| 137 | 1676-1693 | C*G*G*C*A*T*G*T*C*T*A*T*T*T*T |
| | | *G*T*A (OT-101) |
| 138 | — | G*T*T*C*G*T*T*T*A*G*A*G*A*A*C |
| | | *A*G*A*T*C |
| 139 | — | T*A*A*A*G*T*T*C*G*T*T*T*A*G*A |
| | | *G*A*A*C*A*G |
| 140 | — | A*G*C*C*C*T*G*T*A*T*A*C*G*A*C |
| 141 | — | G*T*A*G*G*T*A*A*A*A*A*C*C*T*A |
| | | *A*T*A*T |
| 142 | — | C*G*T*T*T*A*G*A*G*A*A*C*A*G*A |
| | | *T*C*T*A*C |
| 143 | — | C*A*T*T*G*T*A*G*A*T*G*T*C*A*A |
| | | *A*A*G*C*C |
| 144 | — | C*T*C*C*C*T*C*A*T*G*G*T*G*G*C |
| | | *A*G*T*T*G*A |

Embodiments of this invention further include pharmaceutical compositions for inhibiting or suppressing expression of TGF-β, or for treating or ameliorating the symptoms of cancer in a human or animal. The pharmaceutical compositions may contain a TGF-β inhibitor, artemisinin, pharmaceutically acceptable salts forms, esters, polymorphs or stereoisomers thereof, and any combination thereof, as well as a carrier. The TGF-β inhibitor may be selected from TGF-2-specific antisense oligonucleotides. The carrier may be sterile water for injection, saline, isotonic saline, or a combination thereof.

Importantly, a composition of this disclosure may be substantially free of excipients. Compositions of this invention which are substantially free of excipients have been found to be surprisingly stable in a carrier. In some embodiments, the composition may be stable for at least 14 days, or at least 21 days, or at least 28 days in a carrier at 37° C.

In additional embodiments, a pharmaceutical composition for infusion may contain less than 1% by weight of excipients, or less than 0.5% by weight of excipients, or less than 0.1% by weight of excipients.

### OT-101 Antisense Oligonucleotide

The API trabedersen/OT-101 is a synthetic 18-mer S-ODN consisting of the bases adenine (A), thymine (T), guanine (G), and cytosine (C), with all 3′-5′ linkages modified to phosphorothioates. The thioate modification can make the drug more resistant to degradation, resulting in an increased stability in vitro and in vivo. Its molecular structure (nucleotide sequence) can be designed to be complementary to a specific sequence of human transforming growth factor-beta 2 (TGF-β2) mRNA. This sequence can be selected among related molecules for its superior chemical and structural properties, biological activity, and specificity to achieve the best antisense effects in vitro and in vivo.

The chemical structure, exemplary of the phosphorothioate moieties (C-A-G), and the physical characteristics of trabedersen are shown in Table 3.

TABLE 3
Chemical and Physical Characteristics of Trabedersen

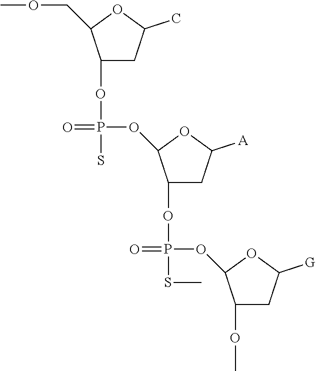

Phosphorothioate linkages
(C-A-G)

| Appearance: | White to off-white solid |
|---|---|
| Molecular Formula (sodium salt, anhydrous): | C₁₇₇N₂₀₈N₆₀O₉₄P₁₇S₁₇Na₁₇ |
| Molecular Weight (sodium salt, anhydrous) | 6143 g/mol |
| Number of moieties: | 18 |
| Backbone characteristics: | Phosphorothioates |
| Polymorphism: | No crystalline morphology in the solid |
| Water content: | Up to 15% |
| Solubility: | Very soluble in water (>50 mg/mL) Soluble in methanol (>8.5 mg/mL) Insoluble in iso-propanol (0.01 mg/mL) |
| pH of solution: | 6-8 |

The IMP can be supplied as a sterile lyophilizate for solution for infusion in 50H glass vials (primary container) containing 7.37 mg trabedersen (intratumoral treatment) and in 20R glass vials (primary container) containing 250 mg trabedersen (intravenous treatment), respectively. No excipients may be in the finished drug product. Glass vials are commonly used for parenterals. Sterile rubber stoppers appropriate for lyophilization can seal the glass vial. The stopper may be sealed with a crimping capsule that includes a colored flip-off cap. For clinical use, each vial can be provided within a white-colored folding box to protect the vials from light exposure and damage during transport. Both the glass vials and the folding boxes can be labeled according to local requirements. The primary and secondary containers of the closure system can fulfill international quality standards for the packaging of sterile solid drug products for injections.

### Immune Checkpoint Inhibitor Agents

As referred to herein, checkpoint inhibitors as known in the art are immune checkpoint inhibitor agents. Checkpoint inhibitors are immunotherapy drugs which block checkpoint proteins from binding with their partner proteins. This prevents an “off” signal from being sent, which allows T cells to kill cancer cells. More particularly, checkpoint proteins, such as PD-1 on T cells, keep immune responses in check. Binding of PD-L1 to PD-1 keeps T cells from killing tumor cells. Thus, blocking the binding of PD-L1 to PD-1 with an immune checkpoint inhibitor may allow the T cells to kill tumor cells. The immune system can be essentially turned back on so that T cells can attack cancer cells.

In some embodiments, a checkpoint inhibitor of this disclosure may be an inhibitor of PD-1, CTLA-4, or PD-L1.

In certain embodiments, a checkpoint inhibitor of this disclosure may be an inhibitor of PD-1.

In certain embodiments, a checkpoint inhibitor of this disclosure may be pembrolizumab.

In certain embodiments, a checkpoint inhibitor of this disclosure may be pembrolizumab, nivolumab, cemiplimab, spartalizumab, atezolizumab, avelumab, or durvalumab.

Without wishing to be bound by theory, the PD-1 receptor-ligand interaction can be a major pathway hijacked by tumors to suppress immune control. The normal function of PD-1, expressed on the cell surface of activated T cells under healthy conditions, is to down-modulate unwanted or excessive immune responses, including autoimmune reactions. Following T-cell stimulation, PD-1 recruits the tyrosine phosphatases, SHP-1 and SHP-2, to the immunoreceptor tyrosine-based switch motif within its cytoplasmic tail, leading to the dephosphorylation of effector molecules such as CD3 zeta (CD3ζ), protein kinase C-theta (PKC0), and zeta-chain-associated protein kinase (ZAP70), which are involved in the CD3 T-cell signaling cascade.

### IL-2 Immunotherapeutic Active Agents

In certain embodiments, an interleukin immunotherapeutic agent of this disclosure may be a natural or synthetic IL-2, a high dose IL-2, a recombinant IL-2, or aldesleukin.

In certain embodiments, an interleukin immunotherapeutic agent of this disclosure can be a recombinant human IL-2 protein.

In additional embodiments, an interleukin immunotherapeutic agent of this disclosure can be an IL-2 protein which is modified to reduce toxic or off-target effects, or is attached to a targeting moiety, or is engineered to target particular cells.

Numbered embodiments of this invention include the following:
(1) A method for treating or ameliorating the symptoms of cancer in a subject in need, the method comprising: administering a composition comprising an agent for inhibiting or suppressing expression of TGF-β2; administering an immune checkpoint inhibitor; and administering an interleukin immunotherapeutic agent to the subject.
(2) An agent for inhibiting or suppressing expression of TGF-β2 in combination with an immune checkpoint inhibitor and an interleukin immunotherapeutic active agent for use in treating or ameliorating the symptoms of cancer.
(3) A composition comprising an agent for inhibiting or suppressing expression of TGF-β2 and a pharmaceutically acceptable carrier for use in the preparation of a medicament or for treating or ameliorating the symptoms of a cancer in a subject in combination with an immune checkpoint inhibitor and an interleukin immunotherapeutic active agent.
(4) The method, agent or composition of any of embodiments 1-3, wherein the cancer is a solid cancer, a pancreatic cancer, melanoma, lung cancer, breast cancer, multiple myeloma, or colorectal cancer.
(5) The method, agent or composition of any of embodiments 1-4, wherein the agent for inhibiting or suppressing expression of TGF-β2, the immune checkpoint inhibitor, and the interleukin immunotherapeutic active agent are administered concurrently, simultaneously, sequentially, or separately in time.
(6) The method, agent or composition of any of embodiments 1-5, wherein the composition and agents are administered by infusion or injection.
(7) The method, agent or composition of any of embodiments 1-6, wherein the agent for inhibiting or suppressing expression of TGF-β2 is selected from Table 1 or Table 2, and chemically-modified variants thereof, LNA variants thereof, gapmer variants thereof, and any combination or pooling thereof.
(8) The method, agent or composition of any of embodiments 1-7, wherein the agent for inhibiting or suppressing expression of TGF-β2 is CGGCATGTCTATTTTGTA SEQ ID NO: 1 or C*G*G*C*A*T*G*T*C*T*A*T*T*T*T*G*T*A SEQ ID NO:137.
(9) The method, agent or composition of any of embodiments 1-8, wherein the agent or composition comprises a carrier of sterile water for injection, saline, isotonic saline, or a combination thereof.
(10) The method, agent or composition of any of embodiments 1-9, wherein the agent or composition is substantially free of excipients.
(11) The method, agent or composition of any of embodiments 1-10, wherein the composition is stable for at least 14 days in carrier at 37° C.
(12) The method, agent or composition of any of embodiments 1-11, wherein the immune checkpoint inhibitor is an inhibitor of PD-1, CTLA-4, or PD-L1.
(13) The method, agent or composition of any of embodiments 1-12, wherein the immune checkpoint inhibitor is pembrolizumab, nivolumab, cemiplimab, spartalizumab, atezolizumab, avelumab, or durvalumab.
(14) The method, agent or composition of any of embodiments 1-13, wherein the interleukin immunotherapeutic active agent is a natural IL-2, a high dose IL-2, a recombinant IL-2, or aldesleukin.
(15) The method, agent or composition of any of embodiments 1-14, wherein the subject upon the administration or use has a reduced TGF-β2 expression.
(16) The method, agent or composition of any of embodiments 1-15, comprising selecting the subjects who benefit from the method, agent or use using one or more biomarkers.
(17) The method, agent or composition of any of embodiments 1-16, wherein the one or more biomarkers are a level of IRF5, a level of ITGAM, or a combination thereof.
(18) The method, agent or composition of any of embodiments 1-17, wherein the one or more biomarkers is ITGAM and the subject is selected when expression of ITGAM is at a level above that found in a healthy patient.
(19) The method, agent or composition of any of embodiments 1-18, wherein the subject after the administration or use has an increased level of IRF5.
(20) The method, agent or composition of any of embodiments 1-19, wherein the subject after the administration or use has an increased level of ITGAM.
(21) The method, agent or composition of any of embodiments 1-20, comprising administering a therapeutically sufficient amount of a pharmaceutical composition comprising an expression product of IRF5 or ITGAM to the subject.
(22) The method, agent or composition of any of embodiments 1-21, wherein the expression product is an mRNA, a polypeptide, a protein, or fragment thereof, or combination thereof.
(23) The method, agent or composition of any of embodiments 1-22, wherein the administration or use decreases mortality rate at month 6, 12, 18, 24, 30, or 36.
(24) The method, agent or composition of any of embodiments 1-23, wherein the administration or use increases survival rate at month 6, 12, 18, 24, 30, or 36.
(25) The method, agent or composition of any of embodiments 1-24, wherein the administration or use of the composition is combined with a standard of care treatment for cancer, wherein the standard of care treatment comprises chemotherapy or radiation therapy.

All publications including patents, patent application publications, and non-patent publications referred to in this description, as well as the sequence listing are each expressly incorporated herein by reference in their entirety for all purposes.

Although the foregoing disclosure has been described in detail by way of example for purposes of clarity of understanding, it will be apparent to the artisan that certain changes and modifications are comprehended by the disclosure and may be practiced without undue experimentation within the scope of the appended claims, which are presented by way of illustration not limitation. This invention includes all such additional embodiments, equivalents, and modifications. This invention includes any combinations or mixtures of the features, materials, elements, or limitations of the various illustrative components, examples, and claimed embodiments.

The designations of agents, compounds and structures of this disclosure are meant to encompass all possible isomers, stereoisomers, diastereomers, enantiomers, and/or optical isomers that would be understood to exist for the specified structure, including any mixture, racemic or otherwise, thereof.

### EXAMPLES

Example 1. This example shows ITGAM is a biomarker for melanoma patients who benefit from a therapeutic combination of a TGF-2-specific antisense agent, an IL-2 immunotherapy agent, and a PD-1 checkpoint inhibitor.

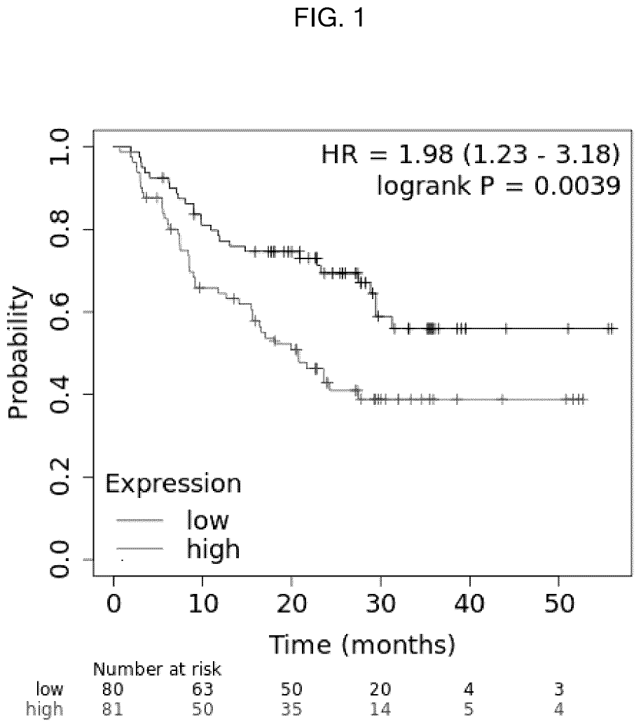

FIG. 1 shows the use of ITGAM as a biomarker for melanoma patients who benefit from a therapeutic combination of a TGF-β2-specific antisense agent, an IL-2 immunotherapy agent, and a PD-1 checkpoint inhibitor.

FIG. 1 shows a Kaplan-Meier overall survival chart obtained in a study of clinical outcomes in melanoma patients (KM Plotter). FIG. 1 shows that for use of a PD-1 checkpoint inhibitor for selected patients with high ITGAM, improved survival was shown for low TGF-β2 (logrank P=0.0039).

Example 2. This example shows ITGAM is a biomarker for pancreatic cancer patients who benefit from a therapeutic combination of a TGF-β2-specific antisense agent, an IL-2 immunotherapy agent, and a PD-1 checkpoint inhibitor.

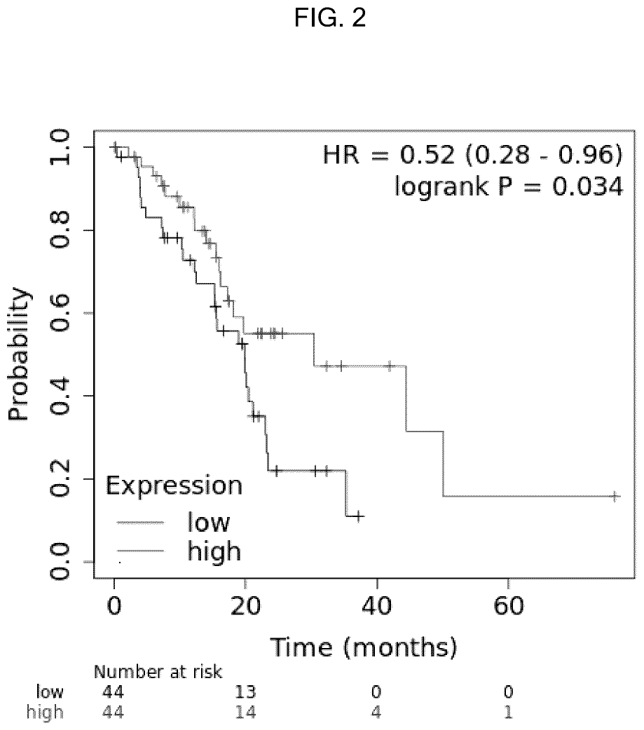

FIG. 2 shows the use of ITGAM as a biomarker for pancreatic cancer patients who benefit from a therapeutic combination of a TGF-β2-specific antisense agent, an IL-2 immunotherapy agent, and a PD-1 checkpoint inhibitor.

FIG. 2 shows a Kaplan-Meier overall survival chart obtained in a study of clinical outcomes in pancreatic cancer (KM Plotter). FIG. 2 shows that for use of IL-2 for selected patients with high ITGAM, improved survival was shown for high IL-2 (logrank P=0.034).

Example 3. This example shows IRF5 is a biomarker for melanoma patients who benefit from a therapeutic combination of a TGF-β2-specific antisense agent, an IL-2 immunotherapy agent, and a PD-1 checkpoint inhibitor.

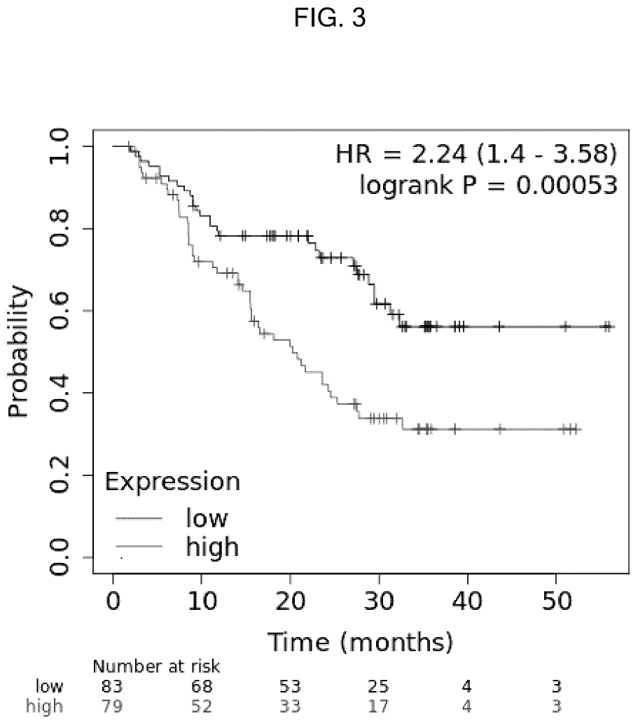

FIG. 3 shows the use of IRF5 as a biomarker for melanoma patients who benefit from a therapeutic combination of a TGF-β2-specific antisense agent, an IL-2 immunotherapy agent, and a PD-1 checkpoint inhibitor.

FIG. 3 shows a Kaplan-Meier overall survival chart obtained in a study of clinical outcomes in melanoma (KM Plotter). FIG. 3 shows that for use of a PD-1 checkpoint inhibitor for selected patients with high IRF5, improved survival was shown for low TGF-β2 (logrank P=0.00053).

Example 4. This example shows that the therapeutic combination of a TGF-β2-specific antisense agent, an IL-2 immunotherapy agent, and a PD-1 checkpoint inhibitor improves survival in cancer.

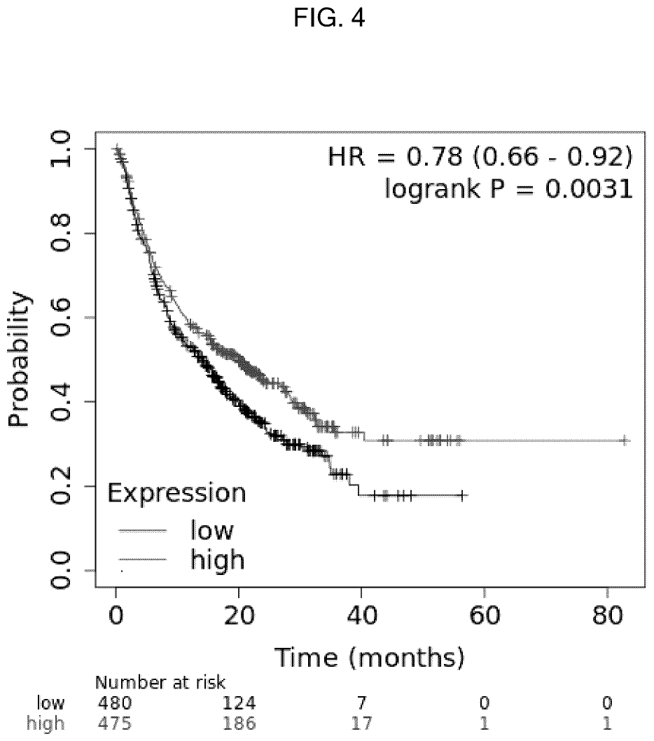

FIG. 4 shows the impact on overall survival in cancer for a therapeutic combination of a TGF-β2-specific antisense agent, an IL-2 immunotherapy agent, and a PD-1 checkpoint inhibitor.

FIG. 4 shows a Kaplan-Meier overall survival chart obtained in a study of clinical outcomes in cancer patients (KM Plotter). The study included multiple kinds of tumors as follows: bladder (N=73), esophageal adenocarcinoma (N=103), glioblastoma (N=28), hepatocellular carcinoma (N=22), HNSCC (N=5), melanoma (N=423), NSCLC (N=21), NSCL (n=22), and urothelial (N-348). FIG. 4 shows that for use of a PD-1 checkpoint inhibitor, improved survival was shown for high ratio of IL-2 to TGF-β2 (IL-2/TGF-β2, logrank P=0.0031).

Referring to FIG. 4, the median overall survival for the high expression cohort, i.e. high IL-2/TGF-β2, was surprisingly increased to 23 months as compared to 10 months for median overall survival of the low expression cohort, i.e. low IL-2/TGF-β2.

This study showed the unexpectedly advantageous impact on overall survival and established a basis for a therapeutic combination in cancer of a TGF-β2-specific antisense agent, an IL-2 immunotherapy agent, and a PD-1 checkpoint inhibitor.

Example 5. This example shows that the therapeutic combination of a TGF-β2-specific antisense agent, an IL-2 immunotherapy agent, and a PD-1 checkpoint inhibitor improves survival in melanoma.

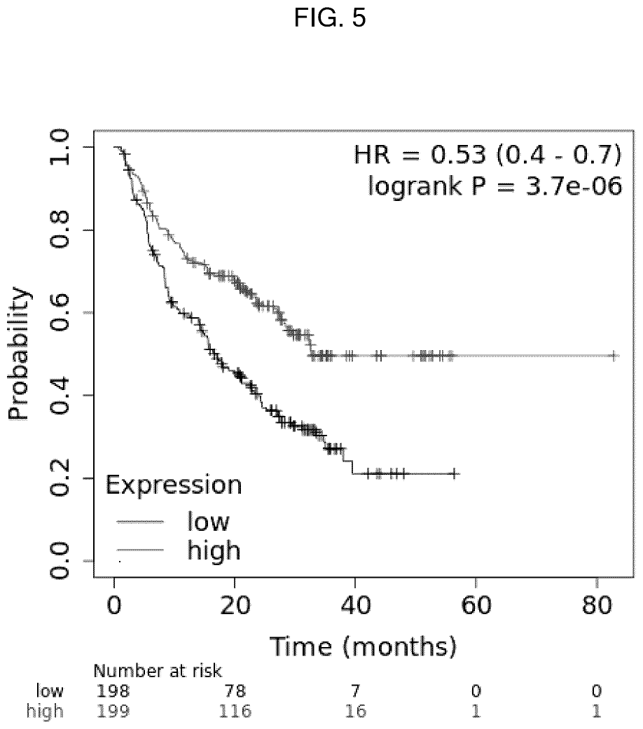

FIG. 5 shows the impact on overall survival in melanoma for a therapeutic combination of a TGF-β2-specific antisense agent, an IL-2 immunotherapy agent, and a PD-1 checkpoint inhibitor.

FIG. 5 shows a Kaplan-Meier overall survival chart obtained in a study of clinical outcomes in melanoma (KM Plotter). FIG. 5 shows that for use of a PD-1 checkpoint inhibitor, improved survival was shown for high ratio of IL-2 to TGF-β2 (IL-2/TGF-β2, logrank P=3.7e-06).

Referring to FIG. 5, the median overall survival for the high expression cohort, i.e. high IL-2/TGF-β2, was surprisingly increased to 31 months as compared to 20 months for median overall survival of the low expression cohort, i.e. low IL-2/TGF-β2.

This study showed the unexpectedly advantageous impact on overall survival and established a basis for a therapeutic combination in melanoma of a TGF-β2-specific antisense agent, an IL-2 immunotherapy agent, and a PD-1 checkpoint inhibitor.

Example 6. This example substantiates that the therapeutic combination of a TGF-β2-specific antisense agent, an IL-2 immunotherapy agent, and a PD-1 checkpoint inhibitor improves survival in melanoma.

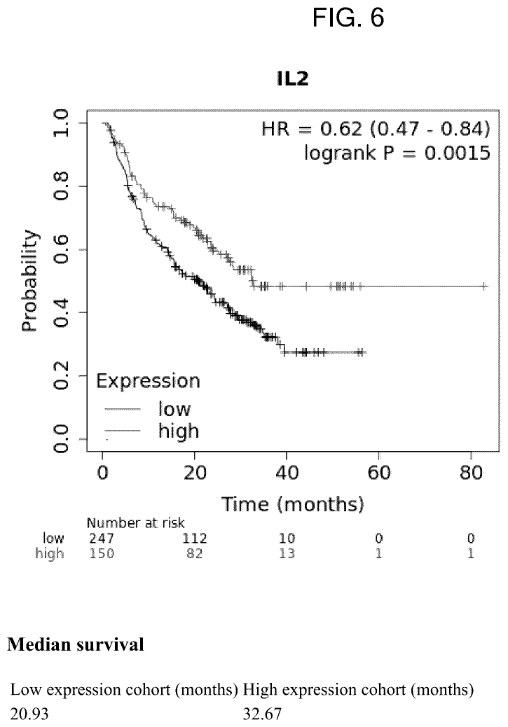

FIG. 6 shows a Kaplan-Meier overall survival chart obtained in a study of clinical outcomes in melanoma patients (KM Plotter). FIG. 6 shows that for use of an immune checkpoint inhibitor, improved survival was shown for high IL-2 (logrank P=0.0015).

Example 7. This example substantiates that the therapeutic combination of a TGF-β2-specific antisense agent, an IL-2 immunotherapy agent, and a PD-1 checkpoint inhibitor improves survival in melanoma.

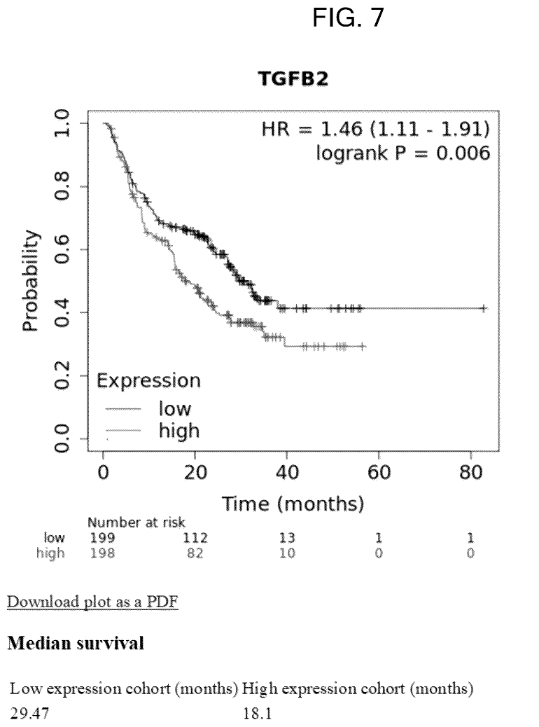

FIG. 7 shows a Kaplan-Meier overall survival chart obtained in a study of clinical outcomes in melanoma patients (KM Plotter). FIG. 7 shows that for use of an immune checkpoint inhibitor, improved survival was shown for low TGF-β2 (logrank P=0.006).

Example 8. This example substantiates that the therapeutic combination of a TGF-β2-specific antisense agent, an IL-2 immunotherapy agent, and a PD-1 checkpoint inhibitor improves survival in cancer.

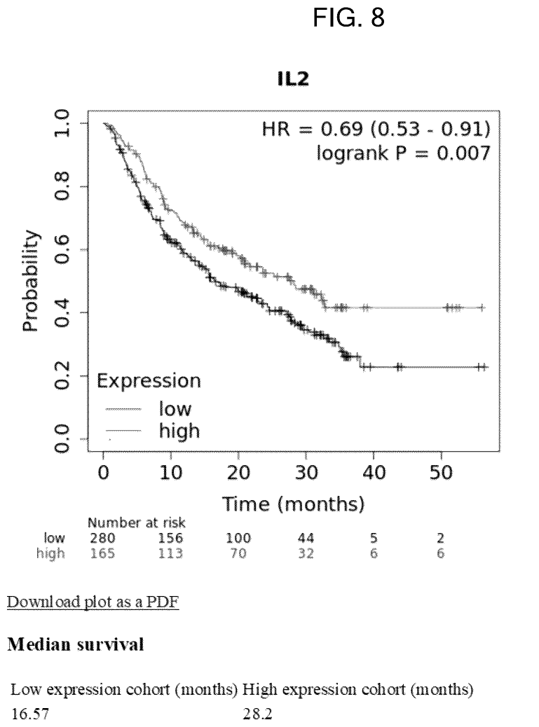

FIG. 8 shows a Kaplan-Meier overall survival chart obtained in a study of clinical outcomes in cancer patients (KM Plotter). FIG. 8 shows that for use of a PD-1 checkpoint inhibitor, improved survival was shown for high IL-2 (logrank P=0.007).

Example 9. This example substantiates that the therapeutic combination of a TGF-β2-specific antisense agent, an IL-2 immunotherapy agent, and a PD-1 checkpoint inhibitor improves survival in cancer.

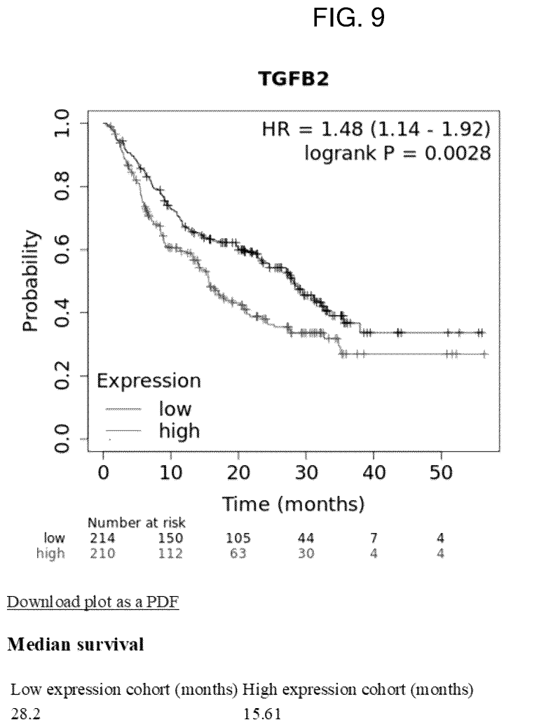

FIG. 9 shows a Kaplan-Meier overall survival chart obtained in a study of clinical outcomes in cancer patients (KM Plotter). FIG. 9 shows that for use of a PD-1 checkpoint inhibitor, improved survival was shown for low TGF-β2 (logrank P=0.0028).

Example 10. This example shows that the therapeutic combination of a TGF-β2-specific antisense agent, an IL-2 immunotherapy agent, and a PD-1 checkpoint inhibitor improves survival in cancer.

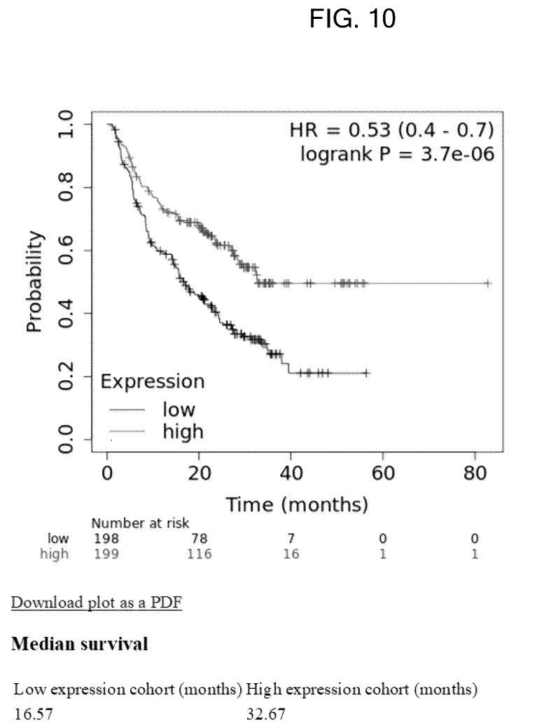

FIG. 10 shows a Kaplan-Meier overall survival chart obtained in a study of clinical outcomes in melanoma patients (KM Plotter). The study included multiple kinds of tumors. FIG. 10 shows that for use of an immune checkpoint inhibitor, improved survival was shown for high ratio of IL-2 to TGF-β2 (IL-2/TGF-β2, logrank P=3.7e-06). Thus, the therapeutic combination of a TGF-β2-specific antisense agent, an IL-2 immunotherapy agent, and a PD-1 checkpoint inhibitor improves survival in cancer.

Example 11. This example shows that the therapeutic combination of a TGF-β2-specific antisense agent, an IL-2 immunotherapy agent, and a PD-1 checkpoint inhibitor improves survival in cancer.

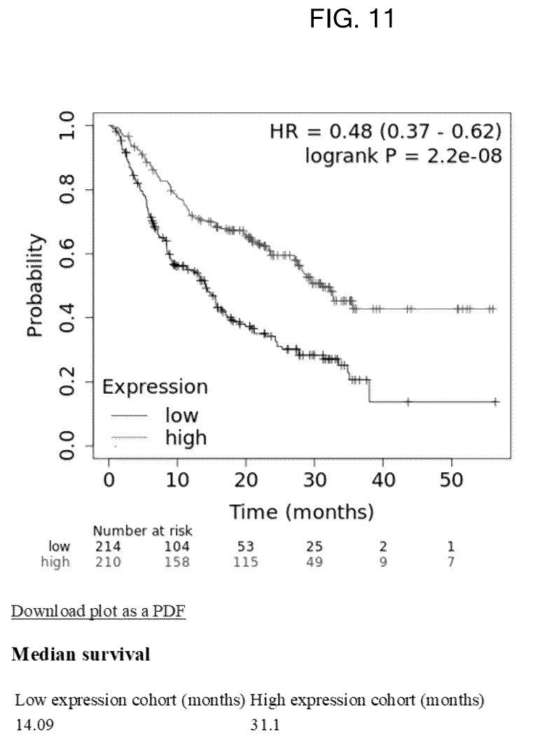

FIG. 11 shows a Kaplan-Meier overall survival chart obtained in a study of clinical outcomes in cancer patients (KM Plotter). FIG. 11 shows that for use of a PD-1 checkpoint inhibitor, improved survival was shown for high ratio of IL-2 to TGF-β2 (IL-2/TGF-β2, logrank P=2.2e-08). Thus, the therapeutic combination of a TGF-β2-specific antisense agent, an IL-2 immunotherapy agent, and a PD-1 checkpoint inhibitor improves survival in cancer.

1. A method for treating or ameliorating the symptoms of cancer in a subject in need, the method comprising:
administering a composition comprising an agent for suppressing expression of TGF-β2;
administering an immune checkpoint inhibitor; and
administering an interleukin immunotherapeutic agent to the subject.

2. (canceled)

3. (canceled)

4. The method of claim 1, wherein the cancer is a solid cancer, a pancreatic cancer, melanoma, lung cancer, breast cancer, multiple myeloma, or colorectal cancer.

5. The method of claim 1, wherein the agent for suppressing expression of TGF-β2, the immune checkpoint inhibitor, and the interleukin immunotherapeutic active agent are administered concurrently, simultaneously, sequentially, or separately in time.

6. The method of claim 1, wherein the composition, inhibitor and interleukin immunotherapeutic agent are administered by infusion or injection.

7. The method of claim 1, wherein the agent for suppressing expression of TGF-β2 is selected from Table 1 or Table 2, and chemically-modified variants thereof, LNA variants thereof, gapmer variants thereof, and any combination or pooling thereof.

8. The method of claim 1, wherein the agent for suppressing expression of TGF-β2 is CGGCATGTCTATTTTGTA SEQ ID NO: 1 or C*G*G*C*A*T*G*T*C*T*A*T*T*T*T*G*T*A SEQ ID NO:137.

9. The method of claim 1, The method, wherein the composition comprises a carrier of sterile water for injection, saline, isotonic saline, or a combination thereof.

10. The method of claim 1, The method, wherein the composition is substantially free of excipients.

11. The method of claim 1, The method, wherein the composition is stable for at least 14 days in carrier at 37° C.

12. The method of claim 1, The method, wherein the immune checkpoint inhibitor is an inhibitor of PD-1, CTLA-4, or PD-L1.

13. The method of claim 1, The method, wherein the immune checkpoint inhibitor is pembrolizumab, nivolumab, cemiplimab, spartalizumab, atezolizumab, avelumab, or durvalumab.

14. The method of claim 1, wherein the interleukin immunotherapeutic active agent is a natural IL-2, a high dose IL-2, a recombinant IL-2, or aldesleukin.

15. The method of claim 1, wherein the subject after all administrations has a reduced TGF-β2 expression.

16. (canceled)

17. The method of claim 1, wherein the subjects have an increased level of IRF5, an increased level of ITGAM, or a combination thereof.

18. The method of claim 1, wherein the subjects have expression of ITGAM at a level above that found in a healthy patient.

19. The method of claim 1, wherein the subjects after all administrations have an increased level of IRF5.

20. The method of claim 1, wherein the subjects after all administrations have an increased level of ITGAM.

21. The method of claim 1, comprising administering a therapeutically sufficient amount of a pharmaceutical composition comprising an expression product of IRF5 or ITGAM to the subject.

22. The method of claim 21, wherein the expression product is an mRNA, a polypeptide, a protein, or fragment thereof, or combination thereof.

23. (canceled)

24. The method of claim 1, wherein the method administration or use increases survival rate at month 6, 12, 18, 24, 30, or 36.

25. The method of claim 1, wherein the method is combined with a standard of care treatment for cancer, wherein the standard of care treatment comprises chemotherapy or radiation therapy.


SEQUENCE LISTING (136 sequences)

SEQ ID NO: 1 - DNA, 18 bp, synthetic construct
  source 1..18: mol_type = other DNA
cggcatgtctattttgta

SEQ ID NO: 2 - DNA, 21 bp, synthetic construct
  source 1..21: mol_type = other DNA
taaagttcgtttagagaacag

SEQ ID NO: 3 - DNA, 15 bp, synthetic construct
  source 1..15: mol_type = other DNA
agccctgtatacgac

SEQ ID NO: 4 - DNA, 19 bp, synthetic construct
  source 1..19: mol_type = other DNA
gtaggtaaaaacctaatat

SEQ ID NO: 5 - DNA, 20 bp, synthetic construct
  source 1..20: mol_type = other DNA
cgtttagagaacagatctac

SEQ ID NO: 6 - DNA, 20 bp, synthetic construct
  source 1..20: mol_type = other DNA
cattgtagatgtcaaaagcc

SEQ ID NO: 7 - DNA, 21 bp, synthetic construct
  source 1..21: mol_type = other DNA
ctccctcatggtggcagttga

SEQ ID NO: 8 - DNA, 20 bp, synthetic construct
  source 1..20: mol_type = other DNA
gttcgtttagagaacagatc

SEQ ID NO: 9 - DNA, 18 bp, synthetic construct
  source 1..18: mol_type = other DNA
tttgttcctggatgactc

SEQ ID NO: 10 - DNA, 18 bp, synthetic construct
  source 1..18: mol_type = other DNA
gtttgttcctggatgact

SEQ ID NO: 11 - DNA, 18 bp, synthetic construct
  source 1..18: mol_type = other DNA
agtttgttcctggatgac

SEQ ID NO: 12 - DNA, 18 bp, synthetic construct
  source 1..18: mol_type = other DNA
cagtttgttcctggatga

SEQ ID NO: 13 - DNA, 18 bp, synthetic construct
  source 1..18: mol_type = other DNA
tcagtttgttcctggatg

SEQ ID NO: 14 - DNA, 18 bp, synthetic construct
  source 1..18: mol_type = other DNA
ctcagtttgttcctggat

SEQ ID NO: 15 - DNA, 18 bp, synthetic construct
  source 1..18: mol_type = other DNA
tgtgtgtgtgtgcgtgtg

SEQ ID NO: 16 - DNA, 18 bp, synthetic construct
  source 1..18: mol_type = other DNA
gtgtgtgtgtgtgcgtgt

SEQ ID NO: 17 - DNA, 18 bp, synthetic construct
  source 1..18: mol_type = other DNA
tgtgtgtgtgtgtgcgtg

SEQ ID NO: 18 - DNA, 18 bp, synthetic construct
  source 1..18: mol_type = other DNA
gtgtgtgtgtgtgtgcgt

SEQ ID NO: 19 - DNA, 18 bp, synthetic construct
  source 1..18: mol_type = other DNA
tgtgtgtgtgtgtgtgcg

SEQ ID NO: 20 - DNA, 18 bp, synthetic construct
  source 1..18: mol_type = other DNA
gtgtgtgtgtgtgtgtgc

SEQ ID NO: 21 - DNA, 18 bp, synthetic construct
  source 1..18: mol_type = other DNA
tgtgtgtgtgtgtgtgtg

SEQ ID NO: 22 - DNA, 18 bp, synthetic construct
  source 1..18: mol_type = other DNA
gtgtgtgtgtgtgtgtgt

SEQ ID NO: 23 - DNA, 18 bp, synthetic construct
  source 1..18: mol_type = other DNA
tgtgtgtgtgtgtgtgtg

SEQ ID NO: 24 - DNA, 18 bp, synthetic construct
  source 1..18: mol_type = other DNA
gtgtgtgtgtgtgtgtgt

SEQ ID NO: 25 - DNA, 18 bp, synthetic construct
  source 1..18: mol_type = other DNA
tgtgtgtgtgtgtgtgtg

SEQ ID NO: 26 - DNA, 18 bp, synthetic construct
  source 1..18: mol_type = other DNA
gtgtgtgtgtgtgtgtgt

SEQ ID NO: 27 - DNA, 18 bp, synthetic construct
  source 1..18: mol_type = other DNA
tgtgtgtgtgtgtgtgtg

SEQ ID NO: 28 - DNA, 18 bp, synthetic construct
  source 1..18: mol_type = other DNA
gtgtgtgtgtgtgtgtgt

SEQ ID NO: 29 - DNA, 18 bp, synthetic construct
  source 1..18: mol_type = other DNA
cgtgtgtgtgtgtgtgtg

SEQ ID NO: 30 - DNA, 18 bp, synthetic construct
  source 1..18: mol_type = other DNA
gcgtgtgtgtgtgtgtgt

SEQ ID NO: 31 - DNA, 18 bp, synthetic construct
  source 1..18: mol_type = other DNA
tgcgtgtgtgtgtgtgtg

SEQ ID NO: 32 - DNA, 18 bp, synthetic construct
  source 1..18: mol_type = other DNA
gtgcgtgtgtgtgtgtgt

SEQ ID NO: 33 - DNA, 18 bp, synthetic construct
  source 1..18: mol_type = other DNA
agtggcggatctgaactc

SEQ ID NO: 34 - DNA, 18 bp, synthetic construct
  source 1..18: mol_type = other DNA
gagtggcggatctgaact

SEQ ID NO: 35 - DNA, 18 bp, synthetic construct
  source 1..18: mol_type = other DNA
gagtgtgctgcaggtaga

SEQ ID NO: 36 - DNA, 18 bp, synthetic construct
  source 1..18: mol_type = other DNA
tcgagtgtgctgcaggta

SEQ ID NO: 37 - DNA, 18 bp, synthetic construct
  source 1..18: mol_type = other DNA
atcgagtgtgctgcaggt

SEQ ID NO: 38 - DNA, 18 bp, synthetic construct
  source 1..18: mol_type = other DNA
gtgctgttgtagatggaa

SEQ ID NO: 39 - DNA, 18 bp, synthetic construct
  source 1..18: mol_type = other DNA
ggtgctgttgtagatgga

SEQ ID NO: 40 - DNA, 18 bp, synthetic construct
  source 1..18: mol_type = other DNA
tggtgctgttgtagatgg

SEQ ID NO: 41 - DNA, 18 bp, synthetic construct
  source 1..18: mol_type = other DNA
ctggtgctgttgtagatg

SEQ ID NO: 42 - DNA, 18 bp, synthetic construct
  source 1..18: mol_type = other DNA
cctggtgctgttgtagat

SEQ ID NO: 43 - DNA, 18 bp, synthetic construct
  source 1..18: mol_type = other DNA
ccctggtgctgttgtaga

SEQ ID NO: 44 - DNA, 18 bp, synthetic construct
  source 1..18: mol_type = other DNA
tccctggtgctgttgtag

SEQ ID NO: 45 - DNA, 18 bp, synthetic construct
  source 1..18: mol_type = other DNA
gtccctggtgctgttgta

SEQ ID NO: 46 - DNA, 18 bp, synthetic construct
  source 1..18: mol_type = other DNA
agtccctggtgctgttgt

SEQ ID NO: 47 - DNA, 18 bp, synthetic construct
  source 1..18: mol_type = other DNA
ctgggttggagatgttaa

SEQ ID NO: 48 - DNA, 18 bp, synthetic construct
  source 1..18: mol_type = other DNA
gctgggttggagatgtta

SEQ ID NO: 49 - DNA, 18 bp, synthetic construct
  source 1..18: mol_type = other DNA
cgctgggttggagatgtt

SEQ ID NO: 50 - DNA, 18 bp, synthetic construct
  source 1..18: mol_type = other DNA
tagcgctgggttggagat

SEQ ID NO: 51 - DNA, 18 bp, synthetic construct
  source 1..18: mol_type = other DNA
atgtagcgctgggttgga

SEQ ID NO: 52 - DNA, 18 bp, synthetic construct
  source 1..18: mol_type = other DNA
agccattcgccttctgct

SEQ ID NO: 53 - DNA, 18 bp, synthetic construct
  source 1..18: mol_type = other DNA
gtctttatggtgaagcca

SEQ ID NO: 54 - DNA, 18 bp, synthetic construct
  source 1..18: mol_type = other DNA
tgtctttatggtgaagcc

SEQ ID NO: 55 - DNA, 18 bp, synthetic construct
  source 1..18: mol_type = other DNA
ctgtctttatggtgaagc

SEQ ID NO: 56 - DNA, 18 bp, synthetic construct
  source 1..18: mol_type = other DNA
cctgtctttatggtgaag

SEQ ID NO: 57 - DNA, 18 bp, synthetic construct
  source 1..18: mol_type = other DNA
gttcctgtctttatggtg

SEQ ID NO: 58 - DNA, 18 bp, synthetic construct
  source 1..18: mol_type = other DNA
ggttcctgtctttatggt

SEQ ID NO: 59 - DNA, 18 bp, synthetic construct
  source 1..18: mol_type = other DNA
aggttcctgtctttatgg

SEQ ID NO: 60 - DNA, 18 bp, synthetic construct
  source 1..18: mol_type = other DNA
caggttcctgtctttatg

SEQ ID NO: 61 - DNA, 18 bp, synthetic construct
  source 1..18: mol_type = other DNA
ccaggttcctgtctttat

SEQ ID NO: 62 - DNA, 18 bp, synthetic construct
  source 1..18: mol_type = other DNA
ggtcttcccactgttttt

SEQ ID NO: 63 - DNA, 18 bp, synthetic construct
  source 1..18: mol_type = other DNA
aggagatgtggggtcttc

SEQ ID NO: 64 - DNA, 18 bp, synthetic construct
  source 1..18: mol_type = other DNA
caggagatgtggggtctt

SEQ ID NO: 65 - DNA, 18 bp, synthetic construct
  source 1..18: mol_type = other DNA
ggttggtctgttgtgact

SEQ ID NO: 66 - DNA, 18 bp, synthetic construct
  source 1..18: mol_type = other DNA
cggttggtctgttgtgac

SEQ ID NO: 67 - DNA, 18 bp, synthetic construct
  source 1..18: mol_type = other DNA
ccggttggtctgttgtga

SEQ ID NO: 68 - DNA, 18 bp, synthetic construct
  source 1..18: mol_type = other DNA
gagaatggttagaggttc

SEQ ID NO: 69 - DNA, 18 bp, synthetic construct
  source 1..18: mol_type = other DNA
gtagagaatggttagagg

SEQ ID NO: 70 - DNA, 18 bp, synthetic construct
  source 1..18: mol_type = other DNA
ggtgttttgccaatgtag

SEQ ID NO: 71 - DNA, 18 bp, synthetic construct
  source 1..18: mol_type = other DNA
gggtgttttgccaatgta

SEQ ID NO: 72 - DNA, 18 bp, synthetic construct
  source 1..18: mol_type = other DNA
tgggtgttttgccaatgt

SEQ ID NO: 73 - DNA, 18 bp, synthetic construct
  source 1..18: mol_type = other DNA
catcatcgttgtcgtcgt

SEQ ID NO: 74 - DNA, 18 bp, synthetic construct
  source 1..18: mol_type = other DNA
gaacggtacgtacagcaa

SEQ ID NO: 75 - DNA, 18 bp, synthetic construct
  source 1..18: mol_type = other DNA
ggaacggtacgtacagca

SEQ ID NO: 76 - DNA, 18 bp, synthetic construct
  source 1..18: mol_type = other DNA
aggaacggtacgtacagc

SEQ ID NO: 77 - DNA, 18 bp, synthetic construct
  source 1..18: mol_type = other DNA
taggaacggtacgtacag

SEQ ID NO: 78 - DNA, 18 bp, synthetic construct
  source 1..18: mol_type = other DNA
ataggaacggtacgtaca

SEQ ID NO: 79 - DNA, 18 bp, synthetic construct
  source 1..18: mol_type = other DNA
gataggaacggtacgtac

SEQ ID NO: 80 - DNA, 18 bp, synthetic construct
  source 1..18: mol_type = other DNA
ggataggaacggtacgta

SEQ ID NO: 81 - DNA, 18 bp, synthetic construct
  source 1..18: mol_type = other DNA
gggataggaacggtacgt

SEQ ID NO: 82 - DNA, 18 bp, synthetic construct
  source 1..18: mol_type = other DNA
gggtgcctattgcatagc

SEQ ID NO: 83 - DNA, 18 bp, synthetic construct
  source 1..18: mol_type = other DNA
agggtgcctattgcatag

SEQ ID NO: 84 - DNA, 18 bp, synthetic construct
  source 1..18: mol_type = other DNA
aagggtgcctattgcata

SEQ ID NO: 85 - DNA, 18 bp, synthetic construct
  source 1..18: mol_type = other DNA
gaagggtgcctattgcat

SEQ ID NO: 86 - DNA, 18 bp, synthetic construct
  source 1..18: mol_type = other DNA
ggaagggtgcctattgca

SEQ ID NO: 87 - DNA, 18 bp, synthetic construct
  source 1..18: mol_type = other DNA
tgggaagggtgcctattg

SEQ ID NO: 88 - DNA, 18 bp, synthetic construct
  source 1..18: mol_type = other DNA
atgggaagggtgcctatt

SEQ ID NO: 89 - DNA, 18 bp, synthetic construct
  source 1..18: mol_type = other DNA
aatgggaagggtgcctat

SEQ ID NO: 90 - DNA, 18 bp, synthetic construct
  source 1..18: mol_type = other DNA
gaatgggaagggtgccta

SEQ ID NO: 91 - DNA, 18 bp, synthetic construct
  source 1..18: mol_type = other DNA
agaatgggaagggtgcct

SEQ ID NO: 92 - DNA, 18 bp, synthetic construct
  source 1..18: mol_type = other DNA
aagaatgggaagggtgcc

SEQ ID NO: 93 - DNA, 18 bp, synthetic construct
  source 1..18: mol_type = other DNA
taagaatgggaagggtgc

SEQ ID NO: 94 - DNA, 18 bp, synthetic construct
  source 1..18: mol_type = other DNA
gtaagaatgggaagggtg

SEQ ID NO: 95 - DNA, 18 bp, synthetic construct
  source 1..18: mol_type = other DNA
agtaagaatgggaagggt

SEQ ID NO: 96 - DNA, 18 bp, synthetic construct
  source 1..18: mol_type = other DNA
gagtaagaatgggaaggg

SEQ ID NO: 97 - DNA, 18 bp, synthetic construct
  source 1..18: mol_type = other DNA
cagactttctcggtcata

SEQ ID NO: 98 - DNA, 18 bp, synthetic construct
  source 1..18: mol_type = other DNA
gcagactttctcggtcat

SEQ ID NO: 99 - DNA, 18 bp, synthetic construct
  source 1..18: mol_type = other DNA
tgcagactttctcggtca

SEQ ID NO: 100 - DNA, 18 bp, synthetic construct
  source 1..18: mol_type = other DNA
atgcagactttctcggtc

SEQ ID NO: 101 - DNA, 18 bp, synthetic construct
  source 1..18: mol_type = other DNA
aatgcagactttctcggt

SEQ ID NO: 102 - DNA, 18 bp, synthetic construct
  source 1..18: mol_type = other DNA
taatgcagactttctcgg

SEQ ID NO: 103 - DNA, 18 bp, synthetic construct
  source 1..18: mol_type = other DNA
gacctggacttttttccc

SEQ ID NO: 104 - DNA, 18 bp, synthetic construct
  source 1..18: mol_type = other DNA
tgacctggacttttttcc

SEQ ID NO: 105 - DNA, 18 bp, synthetic construct
  source 1..18: mol_type = other DNA
ctgacctggacttttttc

SEQ ID NO: 106 - DNA, 18 bp, synthetic construct
  source 1..18: mol_type = other DNA
gctgacctggactttttt

SEQ ID NO: 107 - DNA, 18 bp, synthetic construct
  source 1..18: mol_type = other DNA
ctgcaatgatgtggcaaa

SEQ ID NO: 108 - DNA, 18 bp, synthetic construct
  source 1..18: mol_type = other DNA
tctgcaatgatgtggcaa

SEQ ID NO: 109 - DNA, 18 bp, synthetic construct
  source 1..18: mol_type = other DNA
ttctgcaatgatgtggca

SEQ ID NO: 110 - DNA, 18 bp, synthetic construct
  source 1..18: mol_type = other DNA
cttctgcaatgatgtggc

SEQ ID NO: 111 - DNA, 18 bp, synthetic construct
  source 1..18: mol_type = other DNA
gctgcccacttgcatact

SEQ ID NO: 112 - DNA, 18 bp, synthetic construct
  source 1..18: mol_type = other DNA
gttggcagaacatagaac

SEQ ID NO: 113 - DNA, 18 bp, synthetic construct
  source 1..18: mol_type = other DNA
cgttggcagaacatagaa

SEQ ID NO: 114 - DNA, 18 bp, synthetic construct
  source 1..18: mol_type = other DNA
gcgttggcagaacataga

SEQ ID NO: 115 - DNA, 18 bp, synthetic construct
  source 1..18: mol_type = other DNA
atggggctacaggggata

SEQ ID NO: 116 - DNA, 18 bp, synthetic construct
  source 1..18: mol_type = other DNA
tatggggctacaggggat

SEQ ID NO: 117 - DNA, 18 bp, synthetic construct
  source 1..18: mol_type = other DNA
ttatggggctacagggga

SEQ ID NO: 118 - DNA, 18 bp, synthetic construct
  source 1..18: mol_type = other DNA
agttatggggctacaggg

SEQ ID NO: 119 - DNA, 18 bp, synthetic construct
  source 1..18: mol_type = other DNA
aagttatggggctacagg

SEQ ID NO: 120 - DNA, 18 bp, synthetic construct
  source 1..18: mol_type = other DNA
caagttatggggctacag

SEQ ID NO: 121 - DNA, 18 bp, synthetic construct
  source 1..18: mol_type = other DNA
ccaagttatggggctaca

SEQ ID NO: 122 - DNA, 18 bp, synthetic construct
  source 1..18: mol_type = other DNA
tccaagttatggggctac

SEQ ID NO: 123 - DNA, 18 bp, synthetic construct
  source 1..18: mol_type = other DNA
atccaagttatggggcta

SEQ ID NO: 124 - DNA, 18 bp, synthetic construct
  source 1..18: mol_type = other DNA
tatccaagttatggggct

SEQ ID NO: 125 - DNA, 18 bp, synthetic construct
  source 1..18: mol_type = other DNA
ctatccaagttatggggc

SEQ ID NO: 126 - DNA, 18 bp, synthetic construct
  source 1..18: mol_type = other DNA
attggaggaaatagggtg

SEQ ID NO: 127 - DNA, 18 bp, synthetic construct
  source 1..18: mol_type = other DNA
gtcttgtaggtagcagcc

SEQ ID NO: 128 - DNA, 18 bp, synthetic construct
  source 1..18: mol_type = other DNA
ggtcttgtaggtagcagc

SEQ ID NO: 129 - DNA, 18 bp, synthetic construct
  source 1..18: mol_type = other DNA
tggtcttgtaggtagcag

SEQ ID NO: 130 - DNA, 18 bp, synthetic construct
  source 1..18: mol_type = other DNA
ctggtcttgtaggtagca

SEQ ID NO: 131 - DNA, 18 bp, synthetic construct
  source 1..18: mol_type = other DNA
tctggtcttgtaggtagc

SEQ ID NO: 132 - DNA, 18 bp, synthetic construct
  source 1..18: mol_type = other DNA
gtctggtcttgtaggtag

SEQ ID NO: 133 - DNA, 18 bp, synthetic construct
  source 1..18: mol_type = other DNA
agtctggtcttgtaggta

SEQ ID NO: 134 - DNA, 18 bp, synthetic construct
  source 1..18: mol_type = other DNA
gagtctggtcttgtaggt

SEQ ID NO: 135 - DNA, 18 bp, synthetic construct
  source 1..18: mol_type = other DNA
ggagtctggtcttgtagg

SEQ ID NO: 136 - DNA, 18 bp, synthetic construct
  source 1..18: mol_type = other DNA
aggagtctggtcttgtag